(19) United States
(12) Patent Application Publication
(10) Pub. No.: US 2023/0285040 A1
(43) Pub. Date: Sep. 14, 2023

(54) CLOT RETRIEVAL DEVICE

(71) Applicant: SUZHOU LAVAMED CO., LTD., Jiangsu (CN)

(72) Inventors: Brendan CUNNIFFE, Oranmore (IE); Xin HUA, Suzhou (CN); Quan SHEN, Suzhou (CN)

(21) Appl. No.: 18/018,486

(22) PCT Filed: Jul. 26, 2021
(86) PCT No.: PCT/CN2021/108428

§ 371 Date: Jan. 27, 2023

(30) Foreign Application Priority Data
Jul. 27, 2020 (WO) PCT/CN2020/104855

(51) Int. Cl. A61B 17/221

(52) U.S. Cl. CPC A61B 17/221; A61B 2017/2212; A61B 2017/00778

(57) ABSTRACT

There is provided a clot retrieval device, comprising: a rotator body formed of a plurality of first longitudinal struts, wherein the first longitudinal struts are fixed only at a proximal end and a distal end, and the rotator body is in an expanded configuration when it is in a free state, and can be in a collapsed configuration when it is inserted into a catheter; and a distal body connected to the distal end of the rotator body, the distal body is in an expanded configuration when it is in a free state, and can be in a collapsed configuration when it is inserted into a catheter; wherein at least a portion of each of the first longitudinal struts makes both radial expansion and circumferential rotation during a deployment phase of the rotator body from the collapsed configuration to the expanded configuration.

### FIELD OF THE INVENTION

The present invention relates to clot retrieval technologies, and particularly relates to a clot retrieval device for capturing and retrieving obstructions such as a blood clot or other matter formed or lodged in a cranial artery of a patient.

### BACKGROUND OF THE INVENTION

A blood clot that has been formed or lodged in a cranial artery of a patient can result in an acute ischemic stroke event. This clot will block blood flow in the affected artery and result in irreparable damage to brain tissue leading to significant patient morbidity or even mortality. Existing devices at present that aim to achieve retrieval of such clots and restore blood flow have varying degrees of success. It is known that there exists a variety of textures for the clot material that has formed and this can prove challenging for certain devices. Quite often, a retrieval device will fail to retrieve the entire clot in a single pass, which means the blood perfusion in the affected artery is not restored. And the retrieval device may require a number of passes, which means the retriever is pulled proximally back to the entrance of the aspiration catheter before being re-sheathed in the micro-catheter, advanced through the blood clot again, redeployed and retracted again, in order to retrieve a sufficient quantity of clot to enable blood flow to be restored. All of this takes time and effort on the part of a clinician and lengthens the time when a portion of the patient's brain is in a hypoxic state.

All mechanical thrombectomy devices must advance through the blood clot in a crimped collapsed configuration via a micro-catheter whereupon they exit the micro-catheter, radially expand to a deployed state and engage with the clot. The clot infiltrates through the struts of the clot retriever, thereby allowing the retriever to get a “grip” on the clot so that when the clinician pulls on the retriever, the clot is gripped sufficiently well to allow for its removal. Most clot retrievers engage with the clot through radial expansion upon exit of the micro-catheter. Their effectiveness at infiltrating and gripping the clot is determined primarily by the cell-area size of the resulting stent structure (i.e. the size of the open space between neighboring struts) and the radial-outward force developed by that structure. The clot will be retrieved provided that the force imparted on it by the retriever device is greater than the force acting on it within the vessel, referring to FIG. 1:

Fᵣₑₜᵣᵢₑᵥₐₗ>Fᵣₑₛᵢₛₜₐₙₜ

From the illustration below, it is seen that there are two elements to the force that acts on the clot within the vessel, i.e.

Fᵣₑₜᵣᵢₑᵥₐₗ=F_{friction}+F_{impaction}

Where:

F_{friction} is the force resulting from the “stickiness” of clot to vessel wall.

F_{impaction} is the force due to blood pressure differential (proximal to distal) across the clot.

With current devices, there are two distinct approaches to clot integration and retrieval. Second generation devices (e.g. Medtronic Solitaire, Stryker Trevo), which are now effectively considered the “original” devices, develop radial force to sufficiently penetrate the thrombus and achieve a grip firm enough to hold it as it is being removed by a rolling or dragging action. This effectively traps the clot between the vessel wall and the stent with the clot being effectively “rolled” proximally during stent retraction. The higher the radial force, the greater the gripping force on the clot but so also is the frictional force between the clot and the vessel wall since it is pushed against the vessel wall with greater force. This mechanism for clot retrieval becomes somewhat counter-productive if the radial force of the structure is inherently too high. The third-generation devices (e.g. Cerenovous Embotrap II, MicroVention ERIC) retrieve the clot through more of a pushing action. They are designed with large spaces between the various modules of the stent into which the clot is intended to infiltrate so that the force imparted on the clot is primarily tangential to the vessel wall in the proximal axial direction without the same component of radially outward force acting on the clot material. This serves to limit the component of wall friction force that the clot experiences as a result of deployment of the stent and aims to provide for easier retrieval of the clot.

Regardless of the mechanism of clot retrieval, most of the devices go from their crimped collapsed state (i.e. within the micro-catheter) to their deployed expanded state through a purely radial expansion action as shown in FIG. 2. This means that the struts of the structure infiltrate through the clot material purely in a radially outward motion. There is no potential to act on the clot in anything other than:
in a radial outward direction during deployment of the structure
in an axial direction during retrieval of the structure.

In this regard, it is desirable to have an improved device, so as to overcome some of the drawbacks found in current devices and achieve more efficient clot retrieval.

### SUMMARY OF THE INVENTION

The present invention provides a device for clot retrieval. The device provides for engagement with the clot through a combination of radial and circumferential action during a deployment phase and an axial action during a retrieval phase. The combination of radial and circumferential action is achieved purely as a result of the geometries of the struts and how they return to their near-expanded state upon exit from the micro-catheter.

In one embodiment according to the present invention, there is provided a clot retrieval device, comprising: a rotator body formed of a plurality of first longitudinal struts, wherein the first longitudinal struts are fixed only at a proximal end and a distal end, and the rotator body is in an expanded configuration when it is in a free state, and can be in a collapsed configuration when it is inserted into a catheter; and a distal body connected to the distal end of the rotator body, the distal body is in an expanded configuration when it is in a free state, and can be in a collapsed configuration when it is inserted into a catheter; wherein at least a portion of each of the first longitudinal struts makes both radial expansion and circumferential rotation during a deployment phase of the rotator body from the collapsed configuration to the expanded configuration.

Preferably, the plurality of first longitudinal struts have substantially identical geometries to each other.

Preferably, each of the plurality of first longitudinal struts is of an elongate shape, and has a curvature along the longitudinal direction of the rotator body and a curvature in the circumferential direction around the rotator body.

Preferably, each of the plurality of first longitudinal struts can be formed by the following steps: providing a strut in substantially straight form; curving the strut into a flattened geometry, which is substantially in a plane and has a curved shape that includes at least one peak along the longitudinal direction of the flattened geometry; and wrapping the flattened geometry around a forming mandrel, with the longitudinal direction of the flattened geometry being substantially parallel with the longitudinal direction of the mandrel.

Preferably, the flattened geometry includes 2, 3 or 4 repeated peaks.

Preferably, the flattened geometry is substantially of a sinusoidal wave shape or a triangular wave shape.

Preferably, the forming mandrel has a cross section of circle, ellipse or polygon.

Preferably, the forming mandrel has a consistent cross section.

Preferably, the forming mandrel is tapered with a larger diametral dimension at the proximal end and/or at the distal end.

Preferably, the rotator body is formed of three first longitudinal struts, wherein the plurality of first longitudinal struts are fixed together at both the proximal end and the distal end.

Preferably, the distal body is formed of a plurality of second longitudinal struts, wherein the second longitudinal struts are fixed only at a first end and a second end.

Preferably, the plurality of second longitudinal struts have substantially identical geometries to each other and are arranged in a substantially rotationally symmetric way.

Preferably, each of the plurality of second longitudinal struts is of an elongate shape, and has a curvature along the longitudinal direction of the distal body and a curvature in the circumferential direction around the distal body.

Preferably, each of the plurality of second longitudinal struts is formed by the following steps: providing a strut in substantially straight form; curving the strut into a flattened geometry, which is in a plane and has a curved shape that includes at least one peak along the longitudinal direction of the flattened geometry; and wrapping the flattened geometry around a forming profile, with the longitudinal direction of the flattened geometry being substantially in the same plane as the longitudinal direction of the forming profile.

Preferably, the flattened geometry of the second longitudinal strut includes one peak.

Preferably, the flattened geometry of the second longitudinal strut is substantially of a sinusoidal wave shape.

Preferably, the forming profile is of an ellipsoid forming profile.

Preferably, the number of the second longitudinal struts is larger than that of the first longitudinal struts.

Preferably, the distal body is formed of six second longitudinal struts, and the six second longitudinal struts form a ball-like space therebetween in the expanded configuration.

Preferably, the plurality of second longitudinal struts are fixed together at both the first end and the second end.

Preferably, there are the same number of first longitudinal struts and second longitudinal struts, each of the plurality of first longitudinal struts is integrally formed with the corresponding one of the plurality of second longitudinal struts.

Preferably, the plurality of second longitudinal struts are formed from a shape memory material.

Preferably, the plurality of first longitudinal struts are formed from a shape memory material.

Preferably, the shape memory material is Nitinol.

Preferably, each of the plurality of first longitudinal struts and the plurality of second longitudinal struts is a wire with a diameter of 80 microns.

Preferably, further comprising a push wire connected with the proximal end of the rotator body and a micro-catheter with a passage for accommodating the distal body, the rotator body and the push wire.

Unlike other existing mechanical thrombectomy devices, according to the present invention, the struts in the rotator body engage with the clot in the vessel through a combination of radial expansion and circumferential rotation. The ability of this structure to infiltrate through the clot is significantly greater than existing mechanical thrombectomy devices. Existing devices simply radially expand into the clot and will all, to some degree, result in compression of the clot against the vessel wall. The ability of the clot to infiltrate into these thrombectomy devices is down due to the geometry of that device and the radial force it develops. Too much radial force will result in issues during retrieval of the device. However, by radially expanding and rotating the proposed structure of the present invention will infiltrate through the clot using two distinct deformation modes and this results in more effective engagement with the clot. Besides, the potential to dislodge the clot from the vessel wall is much greater with the proposed structure than with traditional mechanical thrombectomy devices. A loosened clot will be retrieved much easier from the vessel since that initial adhesion to the vessel wall will be disrupted. Once the clot has been dislodged from the vessel wall, it will be retained by the struts of the rotator body or it will be retained by the much denser distal body.

### BRIEF DESCRIPTION OF THE DRAWINGS

FIG. 1 shows forces acting on a clot in an artery.

FIG. 2 shows purely radial expansion of standard stent structures.

FIG. 3 shows planar view of the clot retrieval device according to one embodiment of the present invention.

FIG. 4 shows isometric view of the clot retrieval device according to one embodiment of the present invention.

FIG. 5 shows axial view of the clot retrieval device according to one embodiment of the present invention.

FIG. 6 shows flattened (planar) geometry of struts used to form rotator body according to one embodiment of the present invention.

FIG. 7 shows planar to cylindrical geometry transformation of the rotator body according to one embodiment of the present invention.

FIG. 8 shows rotational motion during deployment using sectioned view of rotator body according to one embodiment of the present invention.

FIG. 9 shows location of tracker points on rotator body.

FIG. 10 shows quantification of rotational twist as a result of radial deployment.

FIG. 11 shows isometric view of the standalone distal body geometry.

FIG. 12 shows axial view of the standalone distal body geometry.

FIG. 13 shows planar to cylindrical geometry transformation of the distal body.

FIG. 14 shows fully formed distal body over the forming tool.

FIG. 15 shows some examples of shapes of the first longitudinal struts according to the invention.

FIG. 16 shows some examples of cross sections of forming mandrel according to the invention.

FIG. 17 shows some examples of longitudinal profiles of forming mandrel according to the invention.

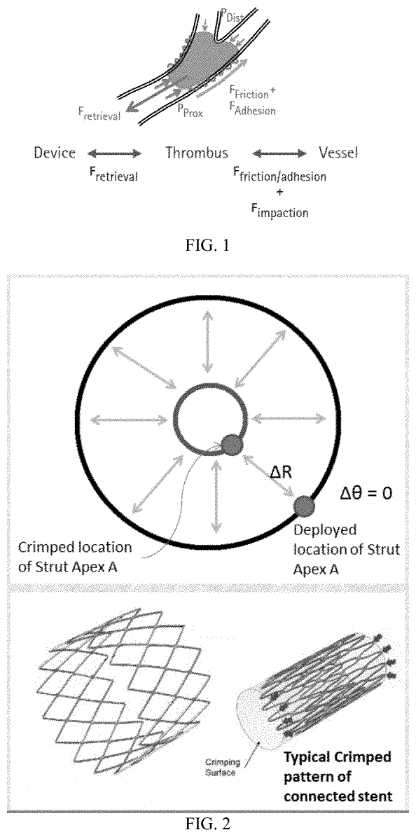

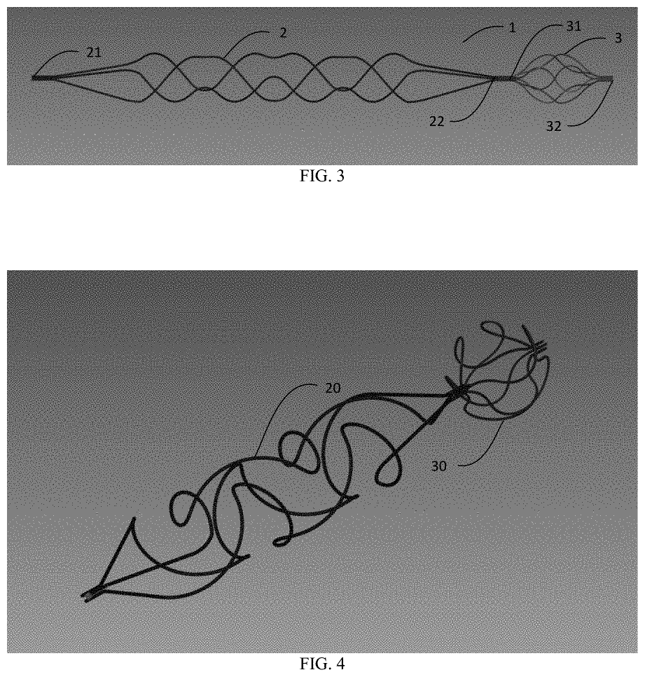

### DETAILED DESCRIPTION OF THE INVENTION

Unless defined otherwise, all technical and scientific terms used herein have the same meanings as commonly understood by one of ordinary skill in the art. Although any methods and materials similar or equivalent to those described herein can be used in the practice or testing of the embodiments described herein, the preferred methods, devices, and materials are described herein.

Specific embodiments of the present invention are now described in detail with reference to the figures, wherein identical reference numbers indicate identical or functionally similar elements. The terms “distal” or “proximal” are used in the following description with respect to a position or direction relative to the treating physician. “Distal” or “distally” is a position distant from or in a direction away from the physician. “Proximal” or “proximally” or “proximate” is a position near or in a direction toward the physician.

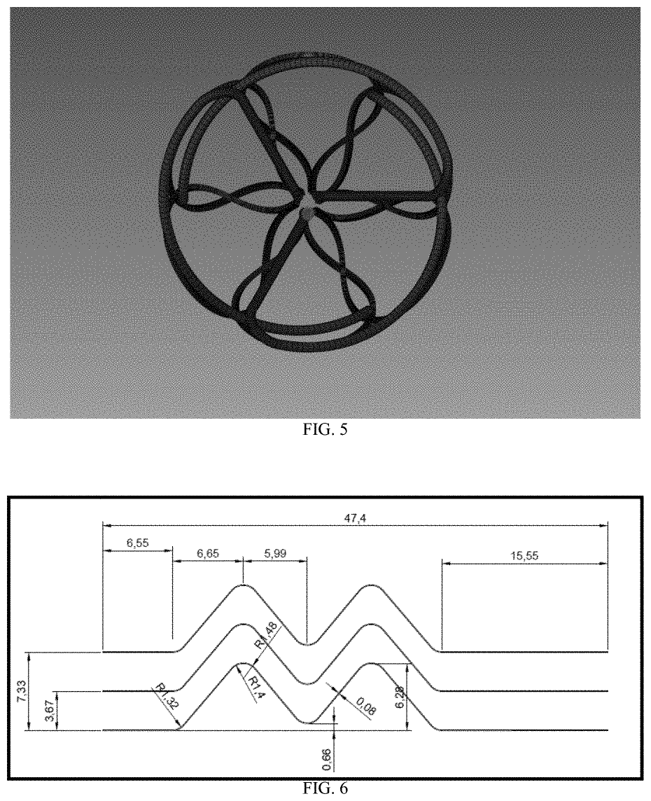

Referring to FIG. 3-5, the present disclosure provides a clot retrieval device (or mechanical thrombectomy device) 1 which comprises a rotator body 2. The rotator body 2 can be in a collapsed configuration for delivery when it is inserted into a catheter or microcatheter and the rotator body 2 is in an expanded configuration when it is in a free state or when it exits the catheter for clot retrieval. The rotator body 2 is formed of a plurality of first longitudinal struts 20, and the first longitudinal struts 20 are fixed only at a proximal end 21 and a distal end 22. At least a portion of each of the first longitudinal struts 20 makes both radial expansion and circumferential rotation during a deployment phase of the rotator body 2 from the collapsed configuration to the expanded configuration. The clot retrieval device 1 also comprises a distal body 3 which is in an expanded configuration when the distal body 3 is in a free state, and can be in a collapsed configuration when the distal body 3 is inserted into a catheter. The distal body 3 is the most distal part of the full structure and it is the portion of the device 1 that is deployed first, before the rotator body 2. The rotator body 2 is a connection between a push wire and the distal body 3.

When talking about rotation and/or angle changes in the context of the present invention, it mainly indicates the rotation of the struts as they “pivot” around the longitudinal axis of the clot retriever. The purpose of the rotation is to dislodge the clot from the vessel wall prior to it being dragged ahead of the “football” feature. It may achieve a substantial loosening effect that the inventors of the present invention are trying to initiate in the clot material so that it is easier to retrieve. It is not found in any other concept or clot retriever on the market that aims to achieve this type of action during the retrieval process.

In one embodiment, referring to FIG. 3-5, the rotator body 2 is formed of three first longitudinal struts 20. The three first longitudinal struts 20 are connected to each other at the proximal end 21 and the distal end 22. The proximal end 21 and the distal end 22 are the only two points where the three first longitudinal struts 20 are connected to each other. Preferably, the three first longitudinal struts 20 are welded together at both the proximal end 21 and the distal end 22 so as to maintain the structure as a whole.

In other embodiments, there may provide more than three first longitudinal struts 20. The first longitudinal struts 20 may be fixed at the proximal end 21 and the distal end 22 in other ways, such as fixed to the push wire at the proximal end 21 and/or fixed with the proximal end of the distal body 3.

The three first longitudinal struts 20 have substantially identical geometries to each other. Each of the plurality of first longitudinal struts 20 is of an elongate shape, and has a curvature along the longitudinal direction of the rotator body 2 and a curvature in the circumferential direction around the rotator body 2.

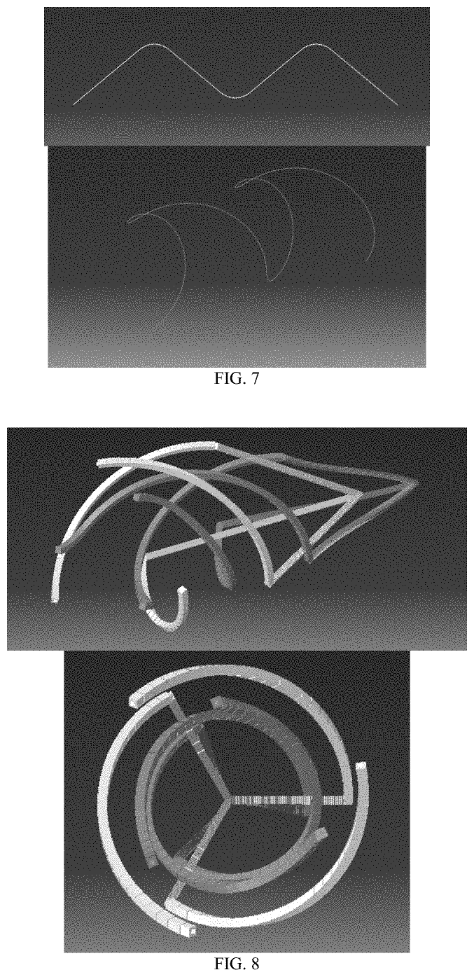

Referring to FIG. 6-7, each of the plurality of first longitudinal struts 20 can be formed by the following steps: providing a strut in substantially straight form; curving the strut into a flattened geometry, as shown in FIG. 7, which is in a plane and has a substantially sinusoidal shape along the longitudinal direction of the flattened geometry; and wrapping the flattened geometry around a cylindrical forming mandrel, with the longitudinal direction of the flattened geometry being parallel with the longitudinal direction of the mandrel.

Specifically, the flattened geometry is a 2D geometry and in general it has a somewhat sinusoidal-like shape. The substantially straight strut is curved into the sinusoidal-like shape substantially in a plane. Optionally, as shown in FIG. 6, the two ends of the struts are still straight, while the middle part of the strut is curved into the sinusoidal-like shape. Two cycles are obtained in the strut through the curving process. There may provide other number of cycles in the flattened geometry of the strut. Preferably, the flattened geometry has a longitudinal direction substantially the same as that of the straight strut. Such sinusoidal-like shape may be called a curvature along the longitudinal direction.

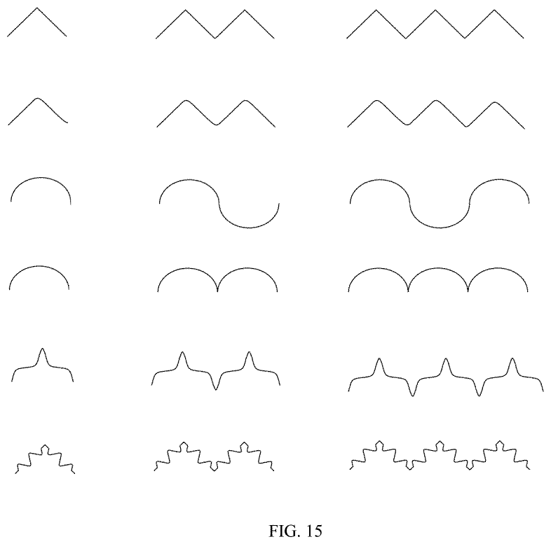

According to other embodiments, the flattened geometry of the strut may be of other shapes, as indicated in FIG. 15. The basic functionality of the rotator body may be achieved by having a “macro” shape that consists of a peak that is held only at the endpoints of the macro shape. There can be a single repeat of this basic peak structure or multiple repeats of the peak structure. This macro shape is then “wrapped” around a cylindrical forming mandrel to give the overall structure of the clot retriever. The overall peak structure does not have to be exclusively sinusoidal in nature. Any structure that has a basic peak structure can produce the same effect. The nature of the shape of the wire between the peaks can be a very simple straight linear connection (as in the top shape above in FIG. 15), curvilinear (as in the second from top shape), circular (or parabolic, as in the third and fourth rows above) or locally sinusoidal (as in the bottom two rows). According to preferable embodiments, the flattened geometry of the first longitudinal strut may be substantially of a sinusoidal wave shape or a triangular wave shape, including 1, 2, 3, 4 or more repeated peaks.

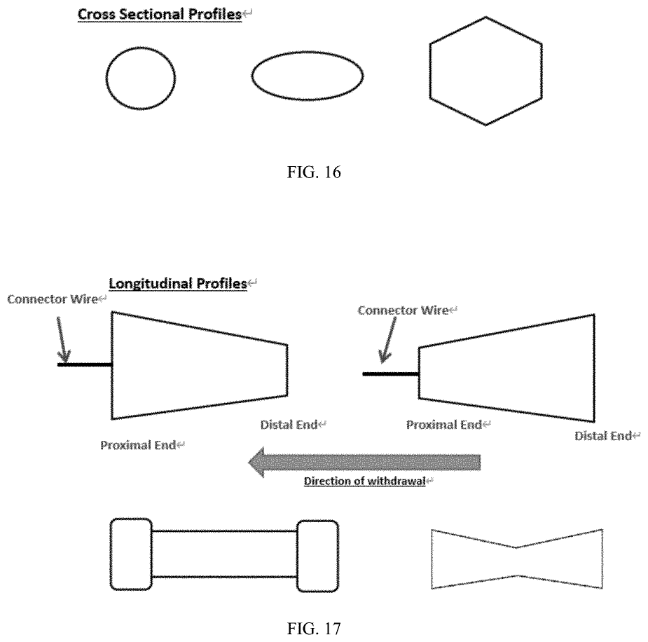

FIG. 16 shows some examples of cross sections of forming mandrel according to the invention. The forming mandrel may have a cross section of circle, ellipse, or polygon. From a practical perspective, the forming cross-sectional profile would need to be essentially cylindrical (or at least elliptical) in nature given that it is to be deployed into a cylindrical artery in the body. From a technical perspective, the forming cross-sectional profile could be polygonal in nature and still produce the rotation action upon deployment.

FIG. 17 shows some examples of longitudinal profiles of forming mandrel according to the invention. For the longitudinal profile, again from a practical perspective it should be essentially cylindrical nature given the nature of the vessel that it is being deployed into. However, it could also be tapered with there being merit in having the tapering biased in either direction:

a) It could be tapered with the larger diametral dimension at the proximal end of the structure so that it tapers in alignment with the natural tapering of the vessels (ie. since the diameter of the vessels decreases as they transition distally).

b) It could be tapered with the larger diametral dimension at the distal end of the structure to ensure that the clot material is pushed ahead of this distal end during retrieval of the clot.

A final configuration of the rotator body would be somewhat “dogbone” in shape.

When pulled into the delivery catheter, these peaks will naturally straighten due to the constraint that the catheter places on the structure. However, the action of deployment (i.e. pushing the structure out of the delivery catheter) removes this constraint and enables the macro structure to return to its formed shape. It is this action of returning to its formed shape that produces the rotation action.

Afterwards, the flattened geometry is wrapped around the cylindrical forming mandrel with the longitudinal direction of the flattened geometry being parallel with the longitudinal direction of the mandrel, and then the shape of the first longitudinal struts 20, as shown in FIG. 7, is obtained. While the flattened geometry is shown as having a sinusoidal-like pattern, in some embodiments, the flattened geometry can have any other suitable curved configuration. Such a curvature obtained by wrapping around the cylindrical forming mandrel may be called a curvature in the circumferential direction.

The method for obtaining the first longitudinal struts 20 is merely an example. It can also be made by other means, such as 3D printing.

In one embodiment of the present invention, three first longitudinal struts are formed by the above steps, and then fixed or welded together at both the proximal end 21 and the distal end 22. Preferably, the three first longitudinal struts are fixed together in a substantially rotationally symmetric way. The proximal end 21 of the rotator body 2 may be connected to a push wire (not shown).

Due to the geometry and the connection pattern of the first longitudinal struts 20, the rotator body 2 can have both radial expansion and circumferential rotation during its deployment phase from the collapsed configuration to the expanded configuration.

In one embodiment, the rotator body 2 is made of a shape-memory material, preferably nitinol, and is self-expandable from the collapsed configuration to the expanded configuration. Any other biocompatible superelastic metallic material is accepetable as well. The rotator body 2 can recover its shape automatically once released from the collapsed strained delivery configuration. The material could be in many forms such as a wire or a tube. The diameter of the wire or the outer diameter of the tube would typically be between 50 microns and 250 microns. In one embodiment, the rotator body 2 is made of round Nitinol wire of approximately 80 microns in diameter. When making the first longitudinal struts 20, any process suitable for shaping a shape-memory material may be used accordingly.

The rotator body 2 can have various lengths and diameters. In one embodiment, the flattened geometry of the rotator body 2 can have length as 47.4 mm as shown in FIG. 6, measured proximally to distally along the longitudinal axis, though other ranges and sizes are also possible. The gross diameter of the formed structure will be comparable to the market leading devices, i.e. sized appropriately to cover an arterial diameter range of 2.0 mm to 6.0 mm with working lengths in the range of 20 mm to 40 mm.

Typically, the formed structure of the rotator body 2 will be “over-sized” relative to the vessel in which it is deployed in order to ensure positive pressure engagement with the vessel. A typical oversizing amount would be between 10% and 33%. Therefore, in some embodiments, if the arterial diameter range to be covered is 2.0 mm to 6.0 mm, then the diameter of the cylindrical forming mandrel would be ranging from 2.2 mm to 8 mm.

The rotator body 2 may be delivered to a required location for retrieval of the blood clot in the cranial artery through a micro-catheter (Not shown). The rotator body 2 is crimped or inserted into the micro-catheter for delivery, being at its collapsed configuration. At the required location in the cranial artery, the rotator body 2 is pushed out of the micro-catheter and, due to the super-elastic nature of Nitinol, it attempts to recover from the deformation that it has experienced during crimping. In other words, it attempts to return to its expanded shape or configuration. When the rotator body 2 recovers its expanded shape automatically from the collapsed configuration, the rotator body 2 infiltrates through the clot, engages with it and retains it during retrieval. Because of the sinusoidal-like geometry of the struts 20 as well as their connection pattern, the rotator body 2 will experience a significant degree of rotational motion during its radial expansion and recovery. So unlike other thrombectomy devices, the rotator body 2 demonstrates both radial and circumferential components of deformation during its deployment phase. This is best illustrated using a finite element model with computer simulation. Therefore, the rotator body 2 can also dislodge the clot from the vessel wall through a rotational action that it exhibits during deployment.

In the finite element model, a sectioned view is taken showing the undeformed expanded and deformed collapsed state, the level of rotational motion in the structure as a result of the deployment can be evaluated and appreciated. Referring to FIG. 8, the first geometry 23 represents the deformed structure whereas the second geometry 24 is the deployed/expanded geometry. A rotation of AO is induced in the structure simply as a result of the radial deployment of the device.

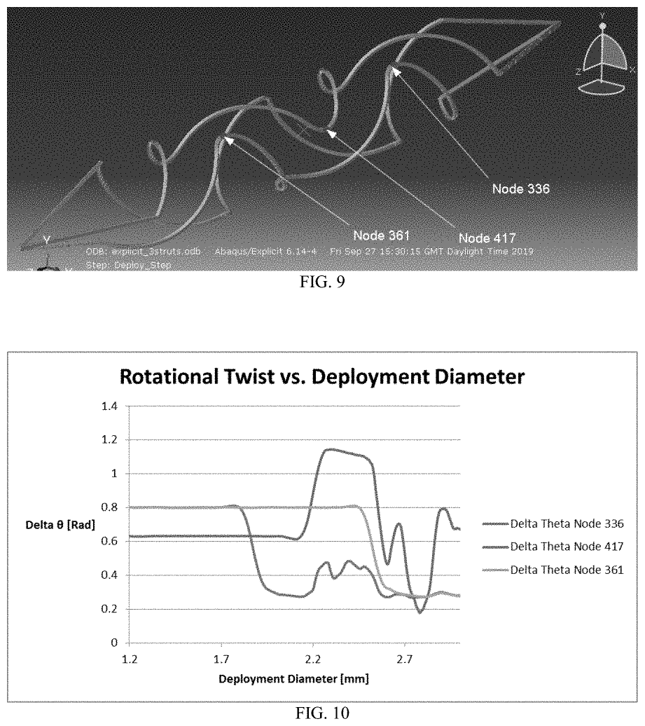

If certain locations in the rotator body 2 are labelled and tracked during this deployment, the magnitude of this rotation can be accurately quantified. Referring to FIG. 9-10, three such locations are identified at various locations in the body and the rotation is graphed as a function of radial change. It is seen that, depending on the location along the rotator body 2, some parts of the structure rotate by up to 65 degrees. It is emphasized again that this rotation occurs organically as a result of the nature of the device's geometry. There is no active loading working on the structure other than it being pushed out of the micro-catheter and returning to its deployed/expanded geometry.

The amount of rotation is related to the degree of circumferential “wrap” that the strut is forced to assume in the forming process. In the geometry according to some embodiments, each sinusoidal flat strut is wrapped around one-half of the circumference of the forming mandrel. So the flat strut is wrapped around 180 degrees of the cylinder. When crimped, it straightens out in order to get into the catheter and in doing so it rotates by a certain degree. When the rotator body is pushed out of the catheter and expands, it rotates in the circumferential direction. Depending on the positions on the rotator body, different parts may rotate for different angles. The region or part that experiences the maximum rotation may be the region or part located furthest from the fixed end points and, in the current configuration, to the maximum rotation angle may be about 65 degrees.

However, if the flat geometry may be wrapped around the mandrel more or less degrees of angle, or the rotator body may be loaded into a smaller or bigger catheter, the rotation angle may be bigger or smaller. Anyway, the rotator body according to the present invention may achieve appropriate rotation angle by properly setting the wrapping angle on the mandrel and/or the size of the catheter. The wrapping angle is preferably 180 degrees, but may be other degrees in some embodiments, such as 30 degrees, 60 degrees, 90 degrees, 120 degrees, 150 degrees, or more than 180 degrees.

In this embodiment, the rotator body 2 engages with the clot through a combination of radial and circumferential action during the deployment phase and an axial action during the retrieval phase. The combination of radial and circumferential action is achieved purely as a result of the geometry of the first longitudinal struts 20 and how they return to their near-expanded state upon exit from the micro-catheter.

The significance of this deformation is that, unlike other mechanical thrombectomy devices, the first longitudinal struts 20 engage with the clot in the vessel through a combination of radial expansion and circumferential rotation. Therefore, the ability of this structure to infiltrate through the clot is significantly greater than existing mechanical thrombectomy devices. Existing devices simply radially expand into the clot and will all, to some degree, result in compression of the clot against the vessel wall. The ability of the clot to infiltrate into these thrombectomy devices is down to the geometry of that device and the radial force it develops. Too much radial force will result in issues during retrieval of the device. However, by radially expanding and rotating the proposed structure will infiltrate through the clot using two distinct deformation modes and this results in more effective engagement with the clot. Besides, due to this structure of the rotator body 2, the potential to dislodge the clot from the vessel wall is much greater with the proposed structure than with traditional mechanical thrombectomy devices. The benefit of this is that a loosened clot will be retrieved much easier from the vessel since that initial adhesion to the vessel wall will be disrupted. Once the clot has been dislodged from the vessel wall, it will be retained by the struts of the rotator body 2 or the distal body 3.

In one preferred embodiment, the distal body 3 is formed of a plurality of second longitudinal struts 30, wherein the second longitudinal struts 30 are fixed only at a first end 31 and a second end 32, and the first end 31 of the distal body 3 is connected to the distal end 22 of the rotator body 2. Preferably, the number of the second longitudinal struts of the distal body is larger than the number of the first longitudinal struts of the rotator body. In one embodiment, the distal body 3 is formed of six second longitudinal struts 30. Thus, the distal body 3 has a much denser matrix than the rotator body 2 when viewed axially. Once the clot has been dislodged from the vessel wall, it will either be retained by the struts of the rotator body 2 or it will be retained by the much denser distal body 3 matrix.

In one embodiment, the distal body 3 and the rotator body 2 are constructed independently as two discrete objects and joined through welding, adhesives, soldering or other robust method. In another embodiment, the struts used to form the rotator body 2 can continue on to form the structure of the distal body 3 as well. There can be a matching number of struts in the distal body 3 as in the rotator body 2. Such a construction would be more robust than any joined configuration but more involved to fabricate.

The first end 31 and the second end 32 are the only two points where the six second longitudinal struts 30 are fixed or connected to each other. In one embodiment, the six second longitudinal struts 30 are welded together at both the first end 31 and the second end 32 so as to maintain the structure as a whole.

The six longitudinal struts 30 have substantially identical geometries to each other. Each of the second longitudinal struts 30 is of an elongate shape, and has a curvature along the longitudinal direction of the distal body 3 and a curvature in the circumferential direction around the distal body 3.

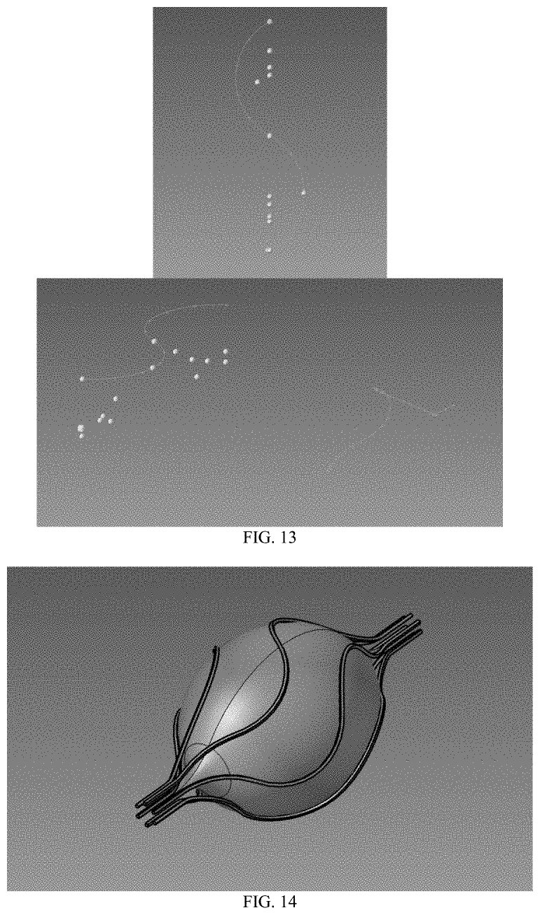

Referring to FIG. 13-14, each second longitudinal struts 30 in distal body 3 can be formed by the following steps: providing a strut in substantially straight form; curving the strut into a flattened geometry, as shown in FIG. 13, which is in a plane and has a substantially sinusoidal shape along the longitudinal direction of the flattened geometry; and wrapping the flattened geometry around an ellipsoid forming profile, with the longitudinal direction of the flattened geometry being substantially in the same plane as the longitudinal direction of the ellipsoid forming profile.

Specifically, the flattened geometry is a 2D geometry. The substantially straight strut is curved into the substantially sinusoidal shape in a plane. Such sinusoidal-like shape may be called a curvature along the longitudinal direction.

Afterwards, the flattened geometry is wrapped around the ellipsoid forming profile, with the longitudinal direction of the flattened geometry being substantially in the same plane as the longitudinal direction of the ellipsoid forming profile, and the shape of the second longitudinal struts 30 is obtained. Such a curvature obtained by wrapping around the ellipsoid forming profile may be called a curvature in the circumferential direction.

The method for obtaining the second longitudinal struts 30 is merely an example. It can also be made by other means, such as 3D printing.

In FIG. 14, each of the six second longitudinal struts 30 are spaced radially equally around the longitudinal direction, or the six second longitudinal struts are fixed together in a substantially rotationally symmetric way. The six second longitudinal struts form a ball-like space therebetween. While the flattened geometry is shown as having a sinusoidal-like pattern, in some embodiments, the flattened geometry can have any other suitable curved configuration. Generally, the shape of the forming profile can include, but are not limited to, oval, elliptical, round, spherical. By spacing the struts evenly around the circumference, a better clot-retention capability may be achieved. In some other embodiments, the second longitudinal struts may be arranged around the circumference in other ways.

According to other embodiments, the distal body 3 may have other shapes and forming ways, like the description of the rotator body 2 mentioned above. In a preferable embodiment, the distal body 3 has fewer peaks in its second longitudinal struts than the first longitudinal structs in the rotator body 2.

Due to the structure of the distal body 3, the distal body 3 also has both radial expansion and circumferential rotation during its deployment phase from the collapsed configuration to the expanded configuration.

The distal body 3 may be also made of a shape-memory material, preferably nitinol, and is self-expandable from the collapsed configuration to the expanded configuration. Any other biocompatible super elastic metallic material is acceptable as well. The distal body 3 can recover its shape automatically once released from a collapsed/strained delivery configuration. The material could be in many forms such as a wire or a tube. The diameter of the wire or the outer diameter of the tube would typically be between 50 microns and 250 microns. When making the second longitudinal struts 30, any process suitable for shaping a shape-memory material may be used accordingly.

In one embodiment, the distal body 3 is made of round Nitinol wire of approximately 80 microns in diameter. The distal body 3 can also have various lengths and diameters. In general, the length of the distal body 3 is shorter than the rotator body 2. The maximum diameter of the distal body 3 will be sized appropriately to cover an arterial diameter range of 2.0 mm to 6.0 mm. Similar with the rotator body 2, the distal body 3 will be “over-sized” relative to the vessel in which it is deployed in order to ensure positive pressure engagement with the vessel. A typical oversizing amount would be between 10% and 33%. Therefore, in some embodiments, if the arterial diameter range to be covered is 2.0 mm to 6.0 mm, then the maximum diameter of the forming profile of the distal body 3 would be ranging from 2.2 mm to 8 mm.

In other embodiments, the distal body 3 may be of a commonly known structure, such as a ball-like mesh structure. When it is combined with the rotator body according to the present invention, it can at least partially solve the problem in the existing devices.

When the device 1 is delivered to a required location for retrieval of the blood clot in the cranial artery, the distal body 3 is deployed in the vessel first since it will be first to exit the micro-catheter. The distal body 3 acts as a distal embolic protection structure, acting to trap any emboli that have become dislodged completely from the primary clot structure and that may attempt to move distally to other vessels within the neurovasculature network. The distal body 3 acts as a “catch-all” structure for the clot retrieval process ensuring that all clot material proximal of the distal body 3 is pushed proximally as the structure is being pulled back towards the aspiration catheter. The distal body 3 provides for a large space between it and the rotator body 2 into which clot can infiltrate and be contained during the retrieval process.

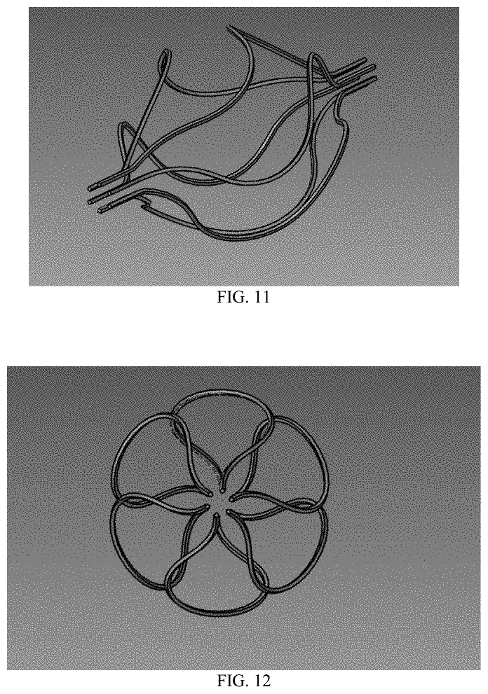

In addition to its role as a distal embolic protection structure, the distal body 3 also serves to act a means of pushing all clot material that is located proximal of it to the aspiration catheter as the entire structure is pulled proximally. Referring to FIG. 12, there are six longitudinal struts 30 comprising the distal body 3 giving a much denser matrix when viewed axially to ensure that its ability to ensure that clot material is not lost distally is as optimized as much as possible. A loosened clot will be retrieved much easier from the vessel since that initial adhesion to the vessel wall will be disrupted. Once the clot has been dislodged from the vessel wall, it will either be retained by the struts of the rotator body 2 or it will be retained by the much denser distal body 3 matrix.

The clot retrieval device may include a push wire connected with the proximal end of the rotator body and a micro-catheter with a passage for accommodating and thus delivering the distal body, the rotator body, and the push wire.

The foregoing description, for purpose of explanation, has been described with reference to specific embodiments according to the present invention. However, the illustrative descriptions above are not intended to be exhaustive or to limit the invention to the precise forms disclosed.

1. A clot retrieval device, comprising:
a rotator body formed of a plurality of first longitudinal struts, wherein the first longitudinal struts are fixed only at a proximal end and a distal end, and the rotator body is in an expanded configuration when it is in a free state, and can be in a collapsed configuration when it is inserted into a catheter; and
a distal body connected to the distal end of the rotator body, the distal body is in an expanded configuration when it is in a free state, and can be in a collapsed configuration when it is inserted into a catheter;
wherein at least a portion of each of the first longitudinal struts makes both radial expansion and circumferential rotation during a deployment phase of the rotator body from the collapsed configuration to the expanded configuration.

2. The clot retrieval device according to claim 1, wherein the plurality of first longitudinal struts have substantially identical geometries to each other and are arranged in a substantially rotationally symmetric way.

3. The clot retrieval device according to claim 2, wherein each of the plurality of first longitudinal struts is of an elongate shape, and has a curvature along a longitudinal direction of the rotator body and a curvature in a circumferential direction around the rotator body.

4. The clot retrieval device according to claim 3, wherein each of the plurality of first longitudinal struts can be formed by following steps:
providing a strut in substantially straight form;
curving the strut into a flattened geometry, which is substantially in a plane and has a curved shape that includes at least one peak along the longitudinal direction of the flattened geometry; and
wrapping the flattened geometry around a forming mandrel, with the longitudinal direction of the flattened geometry being substantially parallel with the longitudinal direction of the mandrel.

5. The clot retrieval device according to claim 4, wherein the flattened geometry includes 2, 3 or 4 repeated peaks; wherein the flattened geometry is substantially of a sinusoidal wave shape or a triangular wave shape.

6. (canceled)

7. The clot retrieval device according to claim 4, the forming mandrel has a cross section of circle, ellipse, or polygon; and
the forming mandrel has a consistent cross section; the forming mandrel is tapered with a larger diametral dimension at the proximal end and/or at the distal end.

8. (canceled)

9. (canceled)

10. The clot retrieval device according to claim 1, wherein the rotator body is formed of three first longitudinal struts, wherein the plurality of first longitudinal struts are fixed together at both the proximal end and the distal end.

11. The clot retrieval device according to claim 1, wherein the distal body is formed of a plurality of second longitudinal struts, wherein the second longitudinal struts are fixed only at a first end and a second end.

12. The clot retrieval device according to claim 11, the plurality of second longitudinal struts have substantially identical geometries to each other and are arranged in a substantially rotationally symmetric way.

13. The clot retrieval device according to claim 12, wherein each of the plurality of second longitudinal struts is of an elongate shape, and has a curvature along a longitudinal direction of the distal body and a curvature in a circumferential direction around the distal body.

14. The clot retrieval device according to claim 13, wherein each of the plurality of second longitudinal struts is formed by following steps:
providing a strut in substantially straight form;
curving the strut into a flattened geometry, which is in a plane and has a curved shape that includes at least one peak along the longitudinal direction of the flattened geometry; and
wrapping the flattened geometry around a forming profile, with the longitudinal direction of the flattened geometry being substantially in same plane as the longitudinal direction of the forming profile.

15. The clot retrieval device according to claim 14, wherein the flattened geometry of the second longitudinal strut includes one peak; wherein the flattened geometry of the second longitudinal strut is substantially of a sinusoidal wave shape; the forming profile is of an ellipsoid forming profile; and wherein a number of the second longitudinal struts is larger than that of the first longitudinal struts.

16. (canceled)

17. (canceled)

18. (canceled)

19. The clot retrieval device according to claim 14, wherein the distal body is formed of six second longitudinal struts, and the six second longitudinal struts form a ball-like space therebetween in the expanded configuration.

20. The clot retrieval device according to claim 14, wherein the plurality of second longitudinal struts are fixed together at both the first end and the second end.

21. The clot retrieval device according to claim 14, wherein there are same number of first longitudinal struts and second longitudinal struts, each of the plurality of first longitudinal struts is integrally formed with the corresponding one of the plurality of second longitudinal struts.

22. The clot retrieval device according to claim 11, wherein the plurality of second longitudinal struts are formed from a shape memory material, wherein the plurality of first longitudinal struts are formed from a shape memory material, the shape memory material is Nitinol.

23. (canceled)

24. (canceled)

25. The clot retrieval device according to claim 11, wherein each of the plurality of first longitudinal struts and the plurality of second longitudinal struts is a wire with a diameter of 80 microns.

26. The clot retrieval device according to claim 1, further comprising a push wire connected with the proximal end of the rotator body and a micro-catheter with a passage for accommodating the distal body, the rotator body, and the push wire.

27. The clot retrieval device according to claim 4, wherein when wrapping the flattened geometry around a forming mandrel, the flattened geometry wraps around the forming mandrel for about 180 degrees.

28. The clot retrieval device according to claim 1, when the rotator body is pushed out of the catheter and turns from the collapsed configuration into the expanded configuration, at least a part of each of the first longitudinal structs rotates for about 65 degrees.
